(19) United States
(12) Patent Application Publication
(10) Pub. No.: US 2011/0280763 A1
(43) Pub. Date: Nov. 17, 2011

(54) LIGHT-ACTIVATED DISINFECTION SYSTEM

(75) Inventors: Stephen L. Trokel, New York, NY (US); Leonard Borrmann, Irvine, CA (US); Chris A. Muller, Foothill Ranch, CA (US); Nicole Bradley, Irvine, CA (US)

(73) Assignee: Abbott Medical Optics Inc., Santa Ana, CA (US)

(21) Appl. No.: 13/109,238

(22) Filed: May 17, 2011

Related U.S. Application Data
(60) Provisional application No. 61/345,483, filed on May 17, 2010.

(51) Int. Cl. A61L 2/18; A61K 31/525

(52) U.S. Cl. 422/24; 514/251

(57) ABSTRACT

The present invention relates to ultraviolet light-activated disinfectants, and more particularly to a light-activated disinfection system for contact lens disinfection.

This application claims priority to U.S. Application No. 61/345,483, filed on May 17, 2010, the entirety of which is hereby incorporated by reference.

### FIELD OF THE INVENTION

The present invention relates to light-activated disinfectants, and more particularly to an light-activated disinfection system for disinfecting contact lenses.

### BACKGROUND OF THE INVENTION

In recent years, increased attention has been paid to the concept of contact lens and lens case disinfection. With the increased prevalence of potentially harmful microorganisms, it is important that all possible care is taken to avoid infection. For example, in recent years several resistant microorganisms forming protective cysts (e.g., Giardia and Cryptosporidium) have been found in drinking water systems. It is believed that these microorganisms have led to some ophthalmic infections. It has been a challenge to adapt conventional contact lens disinfecting techniques such that they are sufficiently efficacious against such organisms.

Contact lenses must be disinfected and cleaned to kill harmful microorganisms that may be present or grow on the lenses, and to remove any buildup that may have accumulated on the lenses. Some of the most popular products for disinfecting lenses are multi-purpose solutions that can be used to clean, disinfect and wet contact lenses, followed by direct insertion (placement on the eye) without rinsing. Obviously, the ability to use a single solution for contact-lens care is an advantage. Such a solution, however, must be particularly gentle to the eye, since at least some of the solution will be on the lens when inserted and will come into contact with the eye.

Adverse changes in ocular tissues during contact lens wear may arise due to exposure of ocular tissues to preservatives, disinfecting agents, cleaning agents and other components in the contact lens care solutions. This can occur through tissue contact with solutions which may directly contact ocular tissues during application or tissue contact with solutions which may adsorb or absorb to the contact lens during treatment of the contact lens by the solution, and subsequently desorb into the eye from the contact lens during wear. This is further complicated by the mechanical effects of placing and wearing a contact lens on the eye.

A significant challenge to improving the disinfecting efficacy of a multi-purpose solution is to simultaneously improve or maintain its contact lens material compatibility and comfort. The addition of more effective disinfecting agents usually has the effect of reducing the material compatibility and comfort of the solution, in particular with silicone and non-silicone soft contact lenses and direct in-eye use. One way to achieve additional material compatibility and comfort is to lower the concentration of a disinfecting agent. However, this heretofore has universally resulted in lower antimicrobial efficacy. Also, it is known that polymeric biguanides, though chemically stable, can become partially depleted in solution over time due to sorption by the container walls, hence requiring a limited shelf life when used at relatively low concentrations that are preferred for comfort reasons.

Riboflavin is an essential human nutrient. Studies in the 1960's and 70's demonstrated that riboflavin exposed to UV-light could inactivate viruses and bacteria. Riboflavin is known to intercalate between the bases of DNA or RNA; light activated riboflavin oxidizes guanine in nucleic acids, preventing replication of the pathogen genome resulting in cell death. Photochemical inactivation of bacteria and viruses has been used in the past to sterilize blood products, and certain pathogens have been shown to be reduced in number with the combination of riboflavin and UV light when tested in the setting of blood products.

It is desirable to develop a disinfectant or disinfecting system which could demonstrate adequate antimicrobial efficacy, yet be sufficiently gentle so that it could be placed on sensitive tissues. It is also desirable to develop a multi-purpose contact-lens solution that would provide increased disinfecting and cleaning efficacy, particularly over time. It would further be desirable to increase the biocidal efficacy of the disinfecting products, including extending the biocidal activity against organisms in both the active, trophzoite phase as well as the protected cyst phase, without adversely affecting material compatibility, ocular comfort or safety in terms of the level of toxicity to sensitive body tissue. Thus, there is a need for a simple product comprising a limited number of antimicrobial agents, at low concentrations, which can achieve disinfection without substantially increasing mammalian cell cytotoxicity and/or discomfort.

### DETAILED DESCRIPTION

It has been discovered that the following naturally occurring compounds: riboflavin, psoralens and porphyrins, when activated by light, provide powerful, yet gentle, disinfection. These activated compounds demonstrate a broad spectrum of antimicrobial activity, including activity against bacteria, viruses, fungi and amoeba. When used herein, the term ‘riboflavin’ includes riboflavin, as well as one or more of the mineral or organic acid addition salts thereof. Similarly, when used herein, the term ‘porphyrin’ includes porphyrins as well as one or more of the mineral or organic acid addition salts thereof.

Riboflavin, psoralens and porphyrins are naturally occurring. Riboflavin, also known as vitamin B2, is an easily absorbed micronutrient with a key role in maintaining health in humans and animals. It is the central component of the cofactors FAD and FMN, and is therefore required by all flavoproteins. As such, vitamin B2 is required for a wide variety of cellular processes. Similarly, porphyrins are a group of chemical compounds, many of which occur in nature. They are heterocyclic macrocycles characterized by the presence of one pyrroline and three pyrrole chemical groups interconnected via their a carbon atoms via methine bridges (═CH—). Psoralens occur naturally in the seeds of Psoralea corylifolia, as well as in the common Fig, celery, parsley and West Indian satinwood. It is widely used in with UV light for treatment of psoriasis, eczema, vitiligo, and Cutaneous T-cell Lymphoma.

Porphyrins are excellent generators of singlet oxygen, and are typically activated in the red end of the spectrum and even the near IR. Without wishing to be bound by theory, the inventors postulate that he porphyrin absorbs light and forms an excited state that can transfer its energy to ground state oxygen. In general, there are at least two mechanisms by which infectious organisms may be killed via the present invention, both of which are a result of generation of singlet oxygen. One of these is the toxicity of the singlet oxygen which is toxic and germicidal. The other is by chemical interaction with the DNA and molecules that may cause molecular disruption or molecular cross linking and enzyme inactivation.

One of ordinary skill in the art will be able to select the appropriate wavelength of light to use with the present invention to achieve the desired level of activity without undue experimentation. For example, riboflavins and psoralens are known to have activity in the uv-range, while porphryins may be activated in the red (610-650 microns), blue (˜410 microns) or near-ir range. Riboflavins also have at least two activation peaks—one about 360-370 microns, and a secondary peak in the blue at about 435 microns. The photoactivation process according to the present invention will reflect the activation spectrum of the selected disinfection entity.

The general technology of an appropriate light source is available over-the-counter from a number of suppliers. Depending on the configuration of the device in accordance with the present invention, such over-the-counter devices may be acceptable; alternatively, it may be preferable to modify the light source from a size or design perspective for functional or aesthetic benefits.

When activated by light and included with the appropriate carrier solution, these compounds provide gentle, thorough disinfection. By way of example, when these compounds are placed in a carrier solution which is then activated by light at the appropriate wavelength, the resulting solution provides disinfection efficacy suitable for disinfecting a contact lens.

A multi-purpose contact lens care solution according to the proposed invention may include a solution containing an appropriate concentration of one of the above compounds and a lens case or other receptacle. Contact lenses would be disinfected when stored in the solution and exposed to the appropriate wavelength light for a period of time, the source of which may or may not be built into the contact lens case. One benefit of the present invention is that light sources are

It is believed that the present invention provides enhanced in-eye safety as manifested by longer contact lens wear times, reduced staining, and reduced hyperemia. It is further believed that these compounds, some of which are naturally occurring in the body, should be less toxic than currently marketed disinfectants. It is further believed that the present invention may be utilized in products to provide (a) faster disinfection time than leading solutions on the market today, which may take 4-6 hours of contact time and (b) disinfection against a broad spectrum of microorganisms.

Compositions according to the present invention may include one or more of the following additional components: additional antimicrobial component(s), surfactant(s), viscosity or thickening agent(s), tonicity agent(s), chelating agent(s) and buffer(s). The additional component or components may be selected from materials which are known to be useful in contact lens care compositions and are included in amounts effective to provide the desired effect or benefit. When an additional component is included, it is generally compatible under typical use and storage conditions with the other components of the composition. For instance, the aforesaid additional component or components are substantially stable in the presence of the antimicrobial and buffer components described herein.

The presently useful additional antimicrobial components include chemicals which derive their antimicrobial activity through a chemical or physiochemical interaction with microbes or microorganisms, such as those contaminating a contact lens. The additional antimicrobial component may be any suitable, preferably ophthalmically acceptable, material effective to disinfect a contact lens contacted with the present solutions or alternatively adequately preserve a solution such as a contact lens rewetting solution.

By way of example, and not of limitation, the additional antimicrobial component may be a monomeric quaternary ammonium or biguanide compound such as chlorhexidine digluconate, chlorhexidine diacetate, benzethonium chloride, myristamidopropyldimethylamine or poly [oxyethylene (dimethyliminio) ethylene-(dimethyliminio) ethylene dichloride] (sold under the trademark WSCP by Buckman Laboratories, Inc.). The additional antimicrobial component may also include, but may not be limited to, quaternary ammonium salts used in ophthalmic applications such as poly [dimethylimino-2-butene-1,4-diyl]chloride, alpha-[4-tris (2-hydroxyethyl)ammonium]-dichloride (chemical registry number 75345-27-6, available under, the trademark Polyquaternium 1® from Onyx Corporation), benzalkonium halides, and biguanides, such as salts of alexidine, alexidine-free base, salts of chlorhexidine, hexamethylene biguanides and their polymers, and salts thereof, antimicrobial polypeptides, chlorine dioxide precursors, and the like and mixtures thereof. Generally, the hexamethylene biguanide polymers (PHMB), also referred to as polyaminopropyl biguanide (PAPB), have molecular weights of up to about 100,000. Such compounds are known and are disclosed in Ogunbiyi et al, U.S. Pat. No. 4,759,595, the disclosure of which is hereby incorporated in its entirety by reference herein.

Generally, the antimicrobial component(s) are present in the liquid aqueous medium at an ophthalmically acceptable or safe concentration such that the user can remove the disinfected lens from the liquid aqueous medium and thereafter directly place the lens in the eye for safe and comfortable wear. Alternatively, the antimicrobial component is present in the liquid aqueous medium at an ophthalmically acceptable or safe concentration and sufficient for maintaining preservative effectiveness. The additional antimicrobial components useful in the present invention preferably are present in the liquid aqueous medium in concentrations in the range of about 0.00001% to about 0.01% (w/v), and more preferably in concentrations in the range of about 0.00005% to about 0.001% (w/v) and most preferably in concentrations in the range of about 0.00005% to about 0.0005% (w/v). Alternatively, the additional antimicrobial component may be present in an amount in the range of from about 0.00001% (w/v) to about 0.0003% (w/v) or about 0.0005% (w/v) or less than 0.005% (w/v). When used in conjunction with the antimicrobials of the present invention, the additional antimicrobial components are typically used at a lower concentration than if the same additional antimicrobial agents were used alone.

The additional antimicrobial components suitable for inclusion in the present invention also include chlorine dioxide precursors. Specific examples of chlorine dioxide precursors include stabilized chlorine dioxide (SCD), metal chlorites, such as alkali metal and alkaline earth metal chlorites, and the like and mixtures thereof. Technical grade sodium chlorite is a very useful chlorine dioxide precursor. Chlorine dioxide containing complexes such as complexes of chlorine dioxide with carbonate, chlorine dioxide with bicarbonate and mixtures thereof are also included as chlorine dioxide precursors. The exact chemical composition of many chlorine dioxide precursors, for example, SCD and the chlorine dioxide complexes, is not completely understood. The manufacture or production of certain chlorine dioxide precursors is described in McNicholas, U.S. Pat. No. 3,278,447, which is incorporated in its entirety herein by reference. Specific examples of useful SCD products include that sold under the trademark Dura Klor® by Rio Linda Chemical Company, Inc., and that sold under the trademark Anthium Dioxide® by International Dioxide, Inc.

If a chlorine dioxide precursor in included in the present compositions, it generally is present in an effective preservative or contact lens disinfecting amount. Such effective preservative or disinfecting concentrations usually are in the range of about 0.002 to about 0.06% (w/v) of the present compositions. The chlorine dioxide precursors may be used in combination with other antimicrobial components, such as biguanides, biguanide polymers, salts thereof and mixtures thereof.

In the event that chlorine dioxide precursors are employed as antimicrobial components, the compositions usually have an osmolality of at least about 200 mOsmol/kg and are buffered to maintain the pH within an acceptable physiological range, for example, a range of about 6 to about 10.

In one embodiment of the present invention, the additional antimicrobial component is non-oxidative. It has been found that reduced amounts of non-oxidative antimicrobial components, for example, in a range of about 0.1 ppm to about 3 ppm or less than 5 ppm (w/v), in the present compositions are effective in disinfecting contact lenses and reduce the risk of such antimicrobial components causing ocular discomfort and/or irritation. Such reduced concentration of antimicrobial component is very useful when the antimicrobial component employed is selected from biguanides, biguanide polymers, salts thereof and mixtures thereof.

The surfactant component generally is present in an amount effective in cleaning, that is to at least facilitate removing, and preferably effective to remove, debris or deposit material from, a contact lens contacted with the surfactant containing solution. Classes of suitable surfactants include poloxamers and poloxamines. Exemplary surfactant components include, but are not limited to, Tetronic 1307, Tetronic 1107, Tetronic 1304, Tetronic 904, Pluronic F87, and mixtures thereof. The amount of surfactant component present, if any, varies over a wide range depending on a number of factors, for example, the concentration of the antimicrobial(s) being used, the specific surfactant or surfactants being used, the other components in the composition and the like. Often the amount of surfactant is in the range of about 0.0003% or about 0.002% to about 0.1% or about 0.5% or about 1.0% (w/v).

By way of further example, and not of limitation, suitable non-ionic surfactants may include block copolymers, tridecyl alcohol ethoxylates, stearyl alcohol ethoxylates, polyethylene glycol esters, octylphenol ethoxylates, nonylphenol ethoxylates, national formulary block copolymers, lauryl alcohol ethoxylates, glycerol esters, ethylene/propylene oxide block copolymers, ethoxylated sorbitan fatty acid esters, decyl alcohol ethoxylates, amine oxides, amine based block copolymers, alcohol ethoxylates, and alcohol alkoxylates.

Any suitable, preferably ophthalmically acceptable viscosity inducing or thickening agent may be included in the present compositions. The viscosity inducing components employed in the present solutions preferably are effective at low or reduced concentrations, compatible with the other components of the present solutions, and anionic and non-ionic. Such viscosity inducing components are effective to enhance and/or prolong the cleaning and wetting activity of the surfactant component and/or condition the lens surface rendering it more hydrophilic (less lipophilic) and/or to act as a demulcent on the eye. Increasing the solution viscosity provides a film on the lens which may facilitate comfortable wearing of the treated contact lens. The viscosity inducing component may also act to cushion the impact on the eye surface during insertion and serves also to alleviate eye irritation. Without wishing to limit the invention to any particular theory of operation, it is believed that the presence of a viscosity inducing component at least assists in providing the lens wearer/user comfort and acceptability benefits of the present invention, which promote regular and consistent contact lens care and ultimately lead to or facilitate better ocular health. The present combinations of components, for example, including such viscosity inducing components, are effective in providing the degree of lens wearer/user comfort and acceptability benefits described herein.

Suitable viscosity inducing components include, but are not limited to, water soluble natural gums, cellulose-derived polymers and the like. Useful natural gums include guar gum, gum tragacanth and the like. Useful cellulose-derived viscosity inducing components include cellulose-derived polymers, such as hydroxypropyl cellulose, hydroxypropylmethyl cellulose, carboxymethyl cellulose, methyl cellulose, hydroxyethyl cellulose and the like. More preferably, the viscosity inducing agent is selected from hyaluronic acid, cellulose derivatives (polymers) and mixtures thereof. A very useful viscosity inducing component is hydroxypropylmethyl cellulose (HPMC).

The viscosity inducing component is used in an amount effective to increase the viscosity of the solution, preferably to a viscosity in the range of about 1.5 to about 30, or even as high as about 750, cps at 25.degree. C., preferably as determined by USP test method No. 911 (USP 23, 1995). To achieve this range of viscosity increase, an amount of viscosity inducing component of about 0.01% to about 5% (w/v) preferably is employed, with amounts of about 0.05% to about 0.5% being more preferred.

The liquid aqueous medium may also include an effective amount of a tonicity component to provide the liquid medium with the desired tonicity. Such tonicity components may be present in the liquid aqueous medium and/or may be introduced into the liquid aqueous medium. Among the suitable tonicity adjusting components that may be employed are those conventionally used in contact lens care products, such as various inorganic salts. Sodium chloride and/or potassium chloride and the like are very useful tonicity components. The amount of tonicity component included is effective to provide the desired degree of tonicity to the solution. Such amount may, for example, be in the range of about 0.1% to about 1.5% (w/v). If a combination of sodium chloride and potassium chloride is employed, it is preferred that the weight ratio of sodium chloride to potassium chloride be in the range of about 2.5 to about 6 or about 8.

The present compositions preferably include a chelating or sequestering component in an amount effective to enhance the effectiveness of the antimicrobial component and/or to complex with metal ions to provide more effective cleaning of the contact lens. A wide range of organic acids, amines or compounds which include an acid group and an amine function are capable of acing as chelating components in the present compositions. For example, nitrilotriacetic acid, diethylenetriaminepentacetic acid, hydroxyethylethylene-diaminetriacetic acid, 1,2-diaminocyclohexane tetraacetic acid, hydroxyethylaminodiacetic acid, ethylenediamine-tetraacetic acid and its salts, polyphosphates, citric acid and its salts, tartaric acid and its salts, and the like and mixtures thereof, are useful as chelating components. Ethylenediaminetetraacetic acid (EDTA) and its alkali metal salts, are preferred, with disodium salt of EDTA, also known as disodium edetate, being particularly preferred. The chelating component preferably is present in an effective amount, for example, in a range of about 0.01% and about 1% (w/v) of the solution.

Any suitable, preferably ophthalmically acceptable buffer component may be included in the present composition. Phosphate, organic amine (e.g., tromethamine) or boric acid buffers are preferred, in an amount effective in maintaining the pH of the composition within a physiologically acceptable range.

The buffer component is present in an amount effective to maintain the pH of the composition or solution in the desired range, for example, in a physiologically acceptable range of about 6 to about 7.5 or about 8.5. In particular, the solution has a pH in the range of about 7 to about 8. The buffer component may include one or more phosphate or tromethamine (TRIS, 2-amino-2-hydroxymethyl-1,3-propanediol) or boric buffers, for example, combinations of monobasic phosphates, dibasic phosphates and the like, or tromethamine and tromethamine hydrochloride. Particularly useful phosphate buffers are those selected from phosphate salts of alkali and/or alkaline earth metals. Examples of suitable phosphate buffers include one or more of sodium phosphate dibasic (Na₂HPO₄) sodium phosphate monobasic (NaH₂PO₄) and potassium phosphate monobasic (KH₂PO₄). The buffer may be a boric acid/sodium hydroxide buffer or a boric acid/sodium borate buffer. The buffer component may also include an amino acid such as taurine. The present buffer components frequently are used in amounts in a range of about 0.01% or about 0.02% to about 0.5% or about 1% (w/v).

Various combinations of two or more of the above noted components may be used in providing at least one of the benefits described herein. Therefore, each and every such combination is included within the scope of the present invention.

In one embodiment, the present compositions comprise: a liquid aqueous medium, riboflavin in an amount effective to, in association with the remainder of the solution when exposed to ultraviolet light at sufficient intensity and for a sufficient period of time, disinfect a contact lens contacted with the composition; a non-ionic surfactant component in an amount effective in cleaning a contact lens contacted with the composition; a buffer component in an amount effective in maintaining the pH of the composition within a physiologically acceptable range; an effective amount of a viscosity inducing component; and an effective amount of a tonicity component. The present compositions may also include an effective amount of a chelating or sequestering component. Each of the components, in the concentration employed, included in the solutions and the formulated solutions of the present invention generally are ophthalmically acceptable. In addition, each of the components in the concentration employed included in the present solutions usually is soluble in the liquid aqueous medium. The solution may also optionally include an additional antimicrobial component in an amount effective to, in association with the remainder of the solution, disinfect a contact lens contacted with the composition.

One of ordinary skill in the art will be able to determine the required concentration of material to be activated as well as the required intensity and time exposure to the appropriate wavelength light which would be needed to provide the required amount of antimicrobial activity.

In one embodiment, the present compositions comprise: a liquid aqueous medium, a porphyrin in an amount effective to, in association with the remainder of the solution when exposed to light at sufficient intensity and for a sufficient period of time, disinfect a contact lens contacted with the composition; a non-ionic surfactant component in an amount effective in cleaning a contact lens contacted with the composition; a buffer component in an amount effective in maintaining the pH of the composition within a physiologically acceptable range; an effective amount of a viscosity inducing component; and an effective amount of a tonicity component. The present compositions may also include an effective amount of a chelating or sequestering component. Each of the components, in the concentration employed, included in the solutions and the formulated solutions of the present invention generally are ophthalmically acceptable. In addition, each of the components in the concentration employed included in the present solutions usually is soluble in the liquid aqueous medium. The solution may also optionally include an additional antimicrobial component in an amount effective to, in association with the remainder of the solution, disinfect a contact lens contacted with the composition.

A solution or component thereof is “ophthalmically acceptable” when it is compatible with ocular tissue, that is, it does not cause significant or undue detrimental effects when brought into contact with ocular tissue. Preferably, each component of the present compositions is also compatible with the other components of the present compositions. The present compositions are more preferably substantially ophthalmically optimized. An ophthalmically optimized composition is one which, within the constraints of component chemistry, minimizes ocular response, or conversely delivers ophthalmic benefit to the lens wearing eye.

When a contact lens is desired to be disinfected by the present compositions, a total amount of antimicrobial component(s) effective to disinfect the lens is used. Generally, such an effective amount of the antimicrobial component reduces the microbial burden or load on the contact lens by one log order in three hours. More preferably, an effective amount of the disinfectant reduces the microbial load by one log order in one hour.

The liquid aqueous medium used is selected to have no substantial deleterious effect on the lens being treated, or on the wearer of the treated lens. The liquid medium is constituted to permit, and even facilitate, the lens treatment or treatments by the present compositions. The liquid aqueous medium advantageously has an osmolality in the range of at least about 175 mOsmol/kg or about 200 mOsmol/kg to about 300 or about 350 mOsmol/kg. The liquid aqueous medium more preferably is substantially isotonic or hypotonic (for example, slightly hypotonic) and/or is ophthalmically acceptable.

Methods for treating a contact lens using the herein described compositions are included within the scope of the invention. Such methods comprise contacting a contact lens with such a composition at conditions effective to provide the desired treatment to the contact lens. Such methods may also include a rubbing step (from about 2 seconds to about 4 or 6 or more seconds per side) and/or a soaking step. The contacting temperature is preferred to be in the range of about 0° C. to about 100° C., and more preferably in the range of about 10° C. to about 60° C., and still more preferably in the range of about 15° C. to about 30° C. Contacting at or about ambient temperature is very convenient and useful. The contacting preferably occurs at or about atmospheric pressure. The contacting preferably occurs for a time in the range of about 5 minutes or about 1 hour to about 8 or about 12 hours or more.

The contact lens can be contacted with the liquid aqueous medium by immersing the lens in the medium. Depending on the structure of the formulation according to the present invention, the activation may occur towards the beginning of the immersion or towards the end of the immersion. For example, where disinfection from the activated compounds is required at the beginning of the immersion time, it may be beneficial to activate the solution at the same time (or shortly after) the lenses are immersed. Alternatively, and perhaps when a second antimicrobial agent is included in the solution, it may be beneficial to activate the disinfecting components according to the present invention at a later point in the immersion cycle/time. This may be important when, for example, there is a chemical interaction between one of the activated disinfecting components according to the present invention and a second component in the solution. If the disinfecting component according to the present invention is activated at a later time, that would allow the second component to provide its beneficial effect prior to activation.

During at least a portion of the contacting, the liquid medium containing the contact lens optionally may be agitated, for example, by shaking the container containing the liquid aqueous medium and contact lens, to at least facilitate removal of deposit material from the lens. Alternatively, or additionally, the solution may be circulated or caused to flow across or around the lens for a desired amount of time. This length of time may be the same as, or more or less than, the time the lens is exposed to the solution. By way of example, the circulation may be from about 15 minutes or about 30 minutes to about 1 hour, about 2 hours or about 4 hours. After such contacting step, the contact lens optionally may be manually rubbed to remove further deposit material from the lens. The cleaning method optionally may also include rinsing the lens substantially free of the liquid aqueous medium prior to returning the lens to a wearer's eye. The rinsing step may be accomplished using the solution formulated according to the present invention.

The present will now be described with regards to some embodiments, though the skilled practitioner will realize that the novel disinfecting compounds according to the present invention may be used in a wider variety of applications.

### Example 1

Several contact lens multi-purpose solutions are formulated by dissolving the ingredients in Table 1 in deionized water. A pair of lenses which have been worn is then placed into a contact lens case which is designed to facilitate the immersion of the lenses in the solution which is formulated according to the present invention. The contact lens case is further designed such that it may expose the solution to the appropriate wavelength light. This exposure may be constant or under the control of the user. The light source may either be in close proximity or incorporated in the lens case. When activated by the user, the light source could be turned on or off either by a switch/button or by closing the lens case.

TABLE 1

| Ingredients | % w/w | % w/w | % w/w | % w/w | % w/w | % w/w |
|---|---|---|---|---|---|---|
| Riboflavin | 0.0015 | 0.003 | | | | |
| porphyrin | | | 0.0015 | 0.003 | | |
| psoralens | | | | | 0.0015 | 0.003 |
| Boric acid | 0.60 | 0.60 | 0.60 | 0.60 | 0.60 | 0.60 |
| Sodium | 0.18 | 0.18 | 0.18 | 0.18 | 0.18 | 0.18 |
| Borate-10H₂O | | | | | | |
| NaCl | 0.40 | 0.40 | 0.40 | 0.40 | 0.40 | 0.40 |
| EDTA | 0.05 | 0.05 | 0.05 | 0.05 | 0.05 | 0.05 |
| Tetronic 904 | 0.10 | 0.10 | 0.10 | 0.10 | 0.10 | 0.10 |
| Pluronic F87 | 0.05 | 0.05 | 0.05 | 0.05 | 0.05 | 0.05 |
| Water | QS | QS | QS | QS | QS | QS |

The above solutions will provide substantial efficacy against the various microbes which may be found on the lenses, but will not be irritating to the eye. Furthermore, the solutions according to the present invention demonstrate reduced staining when compared to some of the currently marked multi-purpose solutions.

The lens case which may be utilized in conjunction with the present invention would need to be made from materials sufficiently resilient to the light and the solutions according to the present invention. The lens case may further be manufactured in such a manner to include a closure system that would prevent leakage. When the light source is incorporated in the lens case, it should be manufactured in such a manner to facilitate a power supply for the light source. Such power supply could be through AC or DC power supplies or batteries (either disposable or rechargeable).

Solutions according to the present invention may be manufactured containing a fixed concentration of either riboflavin or porphyrins (or a mixture thereof) either in multi-dose or unit dose packaging. If the solution is in a multidose configuration, such solution may require the addition of a preservative or second antimicrobial.

The foregoing example demonstrates how the present invention could be used when incorporated in a multi-purpose solution for contact lens care. The activated riboflavin and porphyrins according to the present invention may additionally be used in rewetting and lubricating drops, artificial tears, and pharmaceutical preparations. Furthermore, solutions made according to this process may also be applied to body tissues and exposed to the appropriate wavelength light for sterilization of the operating field prior to surgery or to treat possible infections.

The foregoing written specification is considered to be sufficient to enable one skilled in the art to practice the invention. The foregoing description details certain preferred embodiments of the invention and describes the best mode contemplated by the inventor. It will be appreciated, however, that no matter how detailed the foregoing may appear in text, the invention may be practiced in many ways and the invention should be construed in accordance with the appended claims and any equivalents thereof.

What is claimed is:

1. An ophthalmic multi-purpose solution comprising an antimicrobial component selected from the group consisting of riboflavin, psoralens and porphyrins, wherein the antimicrobial component is exposed to light at a wavelength selected to increase antimicrobial activity of the antimicrobial component.

2. The composition as in claim 1, wherein the ophthalmic multi-purpose solution further comprises an aqueous liquid medium.

3. The composition as in claim 1, wherein the ophthalmic multi-purpose solution further comprises a buffer component.

4. The composition as in claim 1, wherein the ophthalmic multi-purpose solution further comprises a tonicity component.

5. The composition as in claim 1, wherein the ophthalmic multi-purpose solution further comprises a second antimicrobial component.

6. The composition as in claim 1, wherein the ophthalmic multi-purpose solution further comprises a surfactant.

7. The composition as in claim 1, wherein the ophthalmic multi-purpose solution further comprises a viscosity increasing component.

8. A method of treating body tissues prior to a surgical procedure, wherein the method comprises administering a solution to body tissues and then exposing the body tissues to ultraviolet light, the solution comprising the composition of claim 1.

9. A method for treating a contact lens, the method comprising:
contacting the lens with an aqueous solution containing an antimicrobial component selected from the group consisting of riboflavin, psoralens and porphyrins; and
exposing the solution to ultraviolet light.

10. The method as in claim 9, wherein the aqueous solution further comprises an aqueous liquid medium.

11. The method as in claim 9, wherein the aqueous solution further comprises a buffer component.

12. The method as in claim 9, wherein the aqueous solution further comprises a tonicity component.

13. The method as in claim 9, wherein the aqueous solution further comprises a second antimicrobial component.

14. The method as in claim 9, wherein the aqueous solution further comprises a surfactant.

15. The method as in claim 9, wherein the aqueous solution further comprises a viscosity increasing component.
